(19) United States
(12) Patent Application Publication
(10) Pub. No.: US 2025/0281730 A1
(43) Pub. Date: Sep. 11, 2025

(54) COUPLING DEVICE FOR A MEDICAL PUMP

(71) Applicant: W.O.M. WORLD OF MEDICINE GMBH, Berlin (DE)

(72) Inventors: Tobias KLUGE, Berlin (DE); Fabian LANGEN, Berlin (DE); Christian HASER, Stahnsdorf (DE)

(21) Appl. No.: 18/290,565

(22) PCT Filed: May 16, 2022
(86) PCT No.: PCT/IB22/54543

§ 371 Date: May 17, 2024

(30) Foreign Application Priority Data
May 17, 2021 (DE) 10 2021 002 721.3

(51) Int. Cl. A61M 39/12; A61M 13/00; A61M 39/10

(52) U.S. Cl. CPC A61M 39/12; A61M 13/003; A61M 39/1011

(57) ABSTRACT

The present invention relates to a coupling device for medical pumps, in particular for insufflation pumps for endoscopy, by means of which several tube connections can be closed at the same time. On the insufflator side, the coupling device comprises a sleeve-shaped element with insufflation pistons as connecting elements and a tube set compatible with the sleeve-shaped element for connecting a plurality of tubes, wherein the tube set has a plurality of hollow cylinders.

### BACKGROUND OF THE INVENTION

The present invention relates to a coupling device for medical pumps, in particular for insufflation pumps for endoscopy, by means of which several tube connections can be closed at the same time.

### FIELD OF THE INVENTION

During endoscopic examinations and especially during therapeutic interventions, it is well known that the respective body cavity is expanded by the inflow of fluid. During laparoscopy, a gas (preferably CO₂) is usually introduced into the abdomen and an internal pressure is created that is higher than the external pressure. In this way, the abdomen is stretched to create space for the introduction of the surgical equipment. Modern systems also have a suction device in order to be able to quickly remove smoke gases that impair visibility, but also to keep the pressure in the body cavity as constant as possible during the operation.

This poses the problem of connecting the insufflator to various pumps and measuring lines with the necessary tubing. For hygienic reasons, these are disposable items. Connecting several tubes to the insufflator takes time. In addition, there is a risk of mixing up the tubes if the same type of couplings is used. The alternative is to use different coupling systems, which is perceived as cumbersome by the operating staff.

To overcome this technical problem, the coupling device described below is proposed, which can make several tube connections at the same time while ensuring the tightness of each connection.

### SUMMARY OF THE INVENTION

The invention comprises a tube connection device for connecting n tubes, through which a gas flows, to an insufflator, where n is one to five, wherein a sleeve- shaped element is formed on the insufflator side, wherein the sleeve-shaped element has n insufflation pistons as connecting elements in the region of the sleeve base,

wherein the n insufflation pistons as connecting elements protrude from the sleeve base by means of spring tension and can be pressed towards the sleeve base by the pressure from the tubes, whereby the surface of the insufflation pistons is flush with the sleeve base when the tube connection is made,

wherein a resilient sealing element is arranged between each connecting element and each tube, wherein one or more latching elements are arranged in the region of the sleeve opening, which ensure that the tubes engage and lock the tube connection,

wherein connecting elements are arranged on the tube side which are compatible with the connecting elements of the sleeve base,

wherein an element compatible with the sleeve-shaped element has n hollow cylinders as a tube set to which the n tubes are connected so as to ensure a fluid connection of all n tubes to the insufflator.

### DETAILED DESCRIPTION OF THE INVENTION

The advantages of the device according to the invention are particularly apparent when the number of tubes n is two or more.

Therefore, the tube connection device consists of two parts:

A sleeve-shaped part, which is arranged on the insufflator side as a receptacle and a compatible element as a plug-in part, which is inserted into the sleeve-shaped receptacle as a tube set. The sleeve-shaped receptacle has connections to the insufflator on the outside, and n insufflation pistons are arranged inside the sleeve. The plug-in part has connections for n tubes on the outside, and n hollow cylinders compatible with the insufflation pistons of the sleeve part are arranged on the inside.

By combining the resilient sealing elements with an additional and coordinated resilient element (spring) to compensate for positional and tolerance deviations when connecting the tube to the insufflator, tolerances can be compensated for, and a gas-tight connection can be achieved. In this way, larger manufacturing deviations can be tolerated and the cost of high-accuracy components of the tube set can be reduced. Tolerances that are absorbed by the sealing material itself can be increased many times over.

The resilient elements (springs) are designed to initially provide a defined preload to ensure the desired sealing forces. The spring elements have a low spring stiffness so that the counterforce increases only minimally with additional extension.

The combination of resilient elements can be designed separately for each seal to be connected to compensate for additional tolerances due to the geometry of the tube set or different seal contours.

In this way, sealing in several levels can be achieved.

Due to the (axial) displacement of the resilient element, the position of each individual sealing interface can be detected/monitored/evaluated (by microswitches or by means of other position sensors according to the state of the art) and used for:

1.: detection of correct insertion (detection of actuation and correct end position);
2.: identification of the inserted tube set (if, for example, tube set 1 operates different seals than tube set 2).

An alternative design is to connect a plurality of seals with to a single resilient component (a large, resiliently mounted “plate” that contains all the necessary connections).

When assembling the tube receptacle to the device side, additional adjustment of the latching geometry to the seal using setting gauges can be eliminated by selecting the appropriate manufacturing tolerances. The tolerance of the tube-side plug connection can also be increased concerning flatness and position.

In addition to compensating for length tolerances in the seal geometry, the tube set is aligned at the interface prior before the first contact to the seal. This alignment occurs before the tube set first contacts the seal. Alignment prior to seal contact is intended to reduce lateral movement of the seal and seal geometry and increase the seal life. Incorrect insertion is avoided by ensuring that the geometry of the tube set and device is such that they only fit together in one way.

If possible, the tube should be connected to the device with one hand and one handle. This eliminates the need for insertion and removal with subsequent locking.

The total insertion force can be adjusted independently of the seal material by selecting the spring preload and spring characteristics and can be adapted to customer requirements. This means that specific force profiles and haptic feedback can be achieved, for example by using “leading” seals.

The sealing concept of the resilient connections can also be realized for a non-“purely axial” insertion movement. In this case, care must be taken to ensure that the relative movement between the rigid and soft components is minimal/non-critical in order to avoid damage.

The resilient sealing elements can be located/positioned in a variety of ways:

In the preferred design, the rigid sealing contour is positioned in the tube set, and the soft (silicone) seal and spring elements are positioned in the device.

An alternative design is possible, where the soft component (as a wear part) is positioned in the tube set, and the rigid contour is positioned in the device.

In an alternative design, a spring system with a similar characteristic curve can also be integrated into the tube set via an appropriate injection molding design or a separate component.

The tube set has a closed outer contour as a plug-in connection, with the seals of the fluid paths to be connected positioned on the front face. Multiple fluid paths can be connected, and the arrangement of the fluid paths can be freely selected to match the arrangement of the tube receptacle on the device side. In this way, arrangements can be easily realized according to the placement requirements of the tube set as well as the fluid paths or sensors on the device side. A symmetrical arrangement is particularly desirable as it provides the best distribution of forces. However, any other arrangement is possible and does not impair the tolerance-compensating seal according to the invention.

The plug-in connection is made by pushing into the device-side receptacle (sleeve). In the preferred solution, the tubes emerge from the connector laterally to the axis of the connection paths, which has the advantage that they are not bent and kinked by other objects. Kinking would reduce the open cross section of the tube, which would impede the flow. The tubes are pushed inside the plug-in connector onto hollow cylinders and, if necessary, are glued and fluidically connected to the openings in the front face.

The sleeve-shaped receptacle device can be part of the insufflator, for example, recessed into the front or side surface of the insufflator.

When the plug-in connector is connected to the receptacle on the device side, a seal is in front, i.e., making contact, and the rest of the connection is made before the actual locking occurs. This can be used to flush the tubes and blow out the receptacle on the device side. The choice of which seal is the leading one can be freely made and is not predetermined by the features of the invention. The respective tube diameters or diameters of the seals are also freely selectable, but the diameters have an influence on the connection forces and must be designed accordingly.

### BRIEF DESCRIPTION OF THE DRAWINGS

FIG. 1 shows a preferred embodiment of the invention with four tube connections in various views:

Top: Side view with tube set engaged

Bottom: Top view with tube set engaged

FIG. 2 shows again a side view. Three of the tube connections are clearly visible. A fourth connection is hidden in this view.

FIG. 3 shows the top view again. The latching elements near the sleeve opening are clearly visible.

FIGS. 4-8 show figuratively the insertion process and the force-displacement relationship as a graph. The relevant components are highlighted in color in the figures.

FIG. 4 shows the image at the start of compression of the sealing element (contact of the insufflation piston with the tube).

FIG. 5 shows the image at the start of compression of the latching elements (as the coupling closes).

FIG. 6 shows the image when all four springs are compressed.

FIG. 7 shows the locking of the latching elements (latching interlock).

FIG. 8 shows the start of compression of the secondary locking of the latching elements (locking interlock).

FIG. 9 shows one possible force profile. With the points: P1 start of pre-adjustment, P2 end of pre-adjustment, P3 compression of the seal and start of the insufflation piston path, P4 start of closure compression, P5 contact to measuring line, differential pressure and smoke evacuation connection, P6 end of closure compression, P7 compression of all four springs, P8 closure locking, P9 (optional) start of secondary locking compression, P10 start of contact to the rear wall of the sleeve base (receptacle or connector sleeve).

FIG. 10 shows the state of the insertion process for the displacement S at point P3. The relevant components are circled with a dot-dash line.

FIG. 11 shows the state of the insertion process for the displacement S at point P4. The relevant components are circled with a dot-dash line.

FIG. 12 shows the state of the insertion process for the displacement S at point P7. The relevant components are circled with a dot-dash line.

FIG. 13 shows the state of the insertion process for the displacement S at point P8. The relevant components are circled with a dot-dash line.

FIG. 14 shows the state of the insertion process for the displacement S at points P9 and P10. The relevant components are circled with a dashed line for point P9 and a dot-dash line for P10.

FIG. 15 shows a preferred embodiment of the connection of the two elements according to the invention (sleeve on the device side and tube set) with four tube connections in different external views.

Top left: Front view, side view with tube set not engaged (tube set covers the sleeve part/receptacle on the device side).

Top right: Side view with tube set not engaged.

Bottom: Top view with tube set not engaged.

FIG. 16 shows a preferred embodiment of the connection of the two elements according to the invention (sleeve on the device side and tube set) with four tube connections in different views:

Top left: front view, side view with the tube set not engaged (tube set covers the sleeve part/receptacle on the device side), and illustration of the line A-A for the receptacle on the device side.

Top right: side view with tube set not engaged and stepped section of the sleeve part/receptacle on the device side.

Below: top view with tube set not engaged.

FIG. 17 shows a preferred embodiment of the connection of both elements according to the invention (sleeve on the device side and tube set) with four tube connections in a spatial representation with the tube set not engaged with a stepped section of the sleeve part on the device side/receptacle.

FIG. 18 shows a preferred embodiment of the connection of both elements according to the invention (sleeve on the device side and tube set) with four tube connections in a further spatial representation. The tube set connections located on the tube part and adapted to the receptacle are clearly recognizable.

FIG. 19 shows a preferred method of mounting of the tube connection on the device side with four connections in four orientations.

Top left: front view.

Top center: side view.

Top right: rear view.

Bottom: top view.

FIG. 20 shows a preferred embodiment of the tube part with four connections in four orientations.

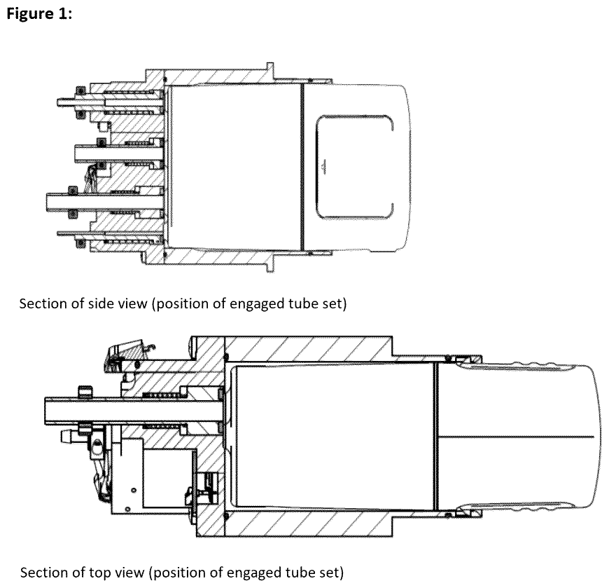

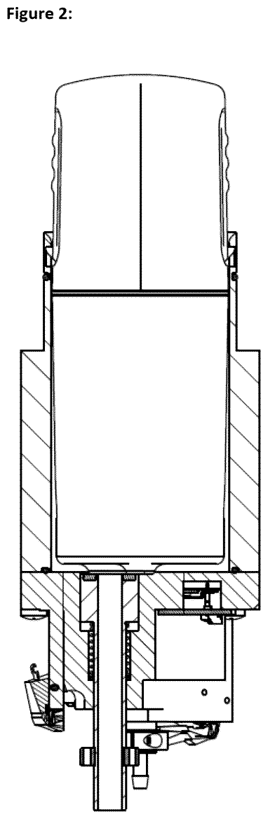

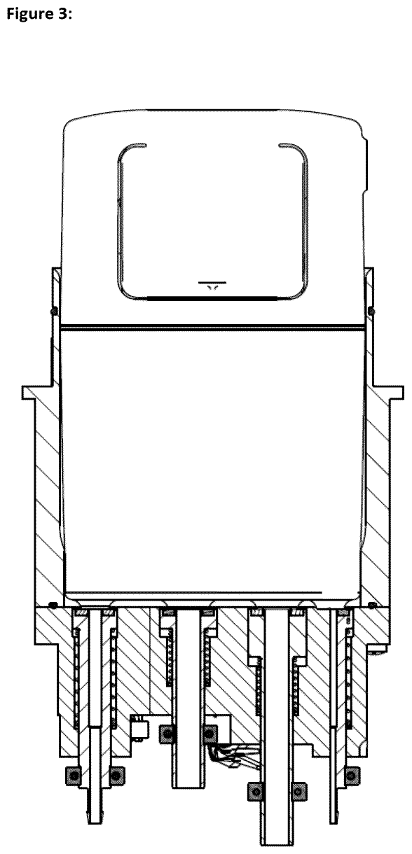

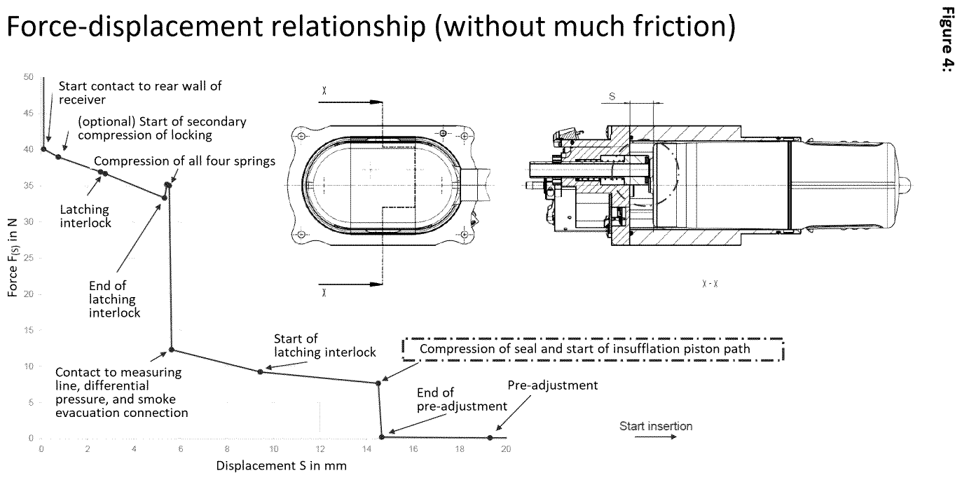

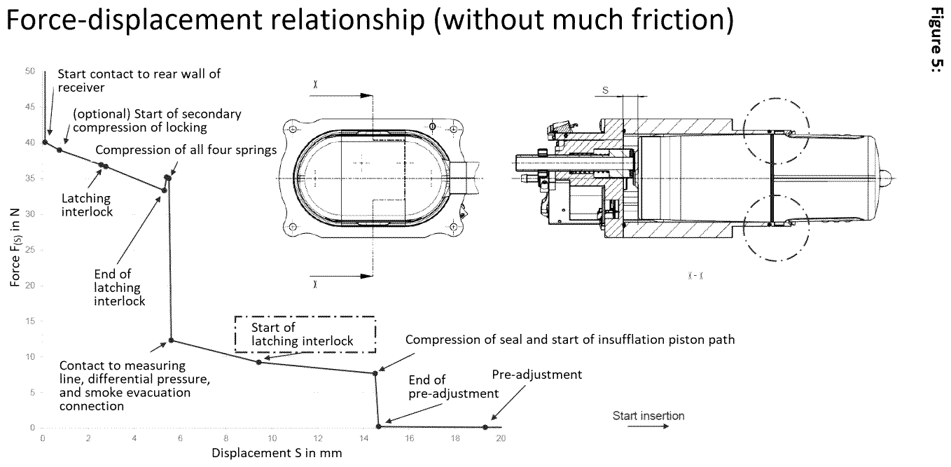

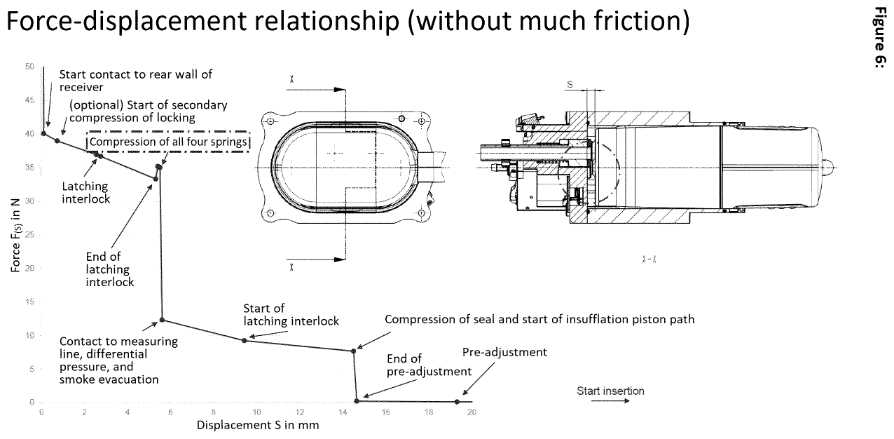

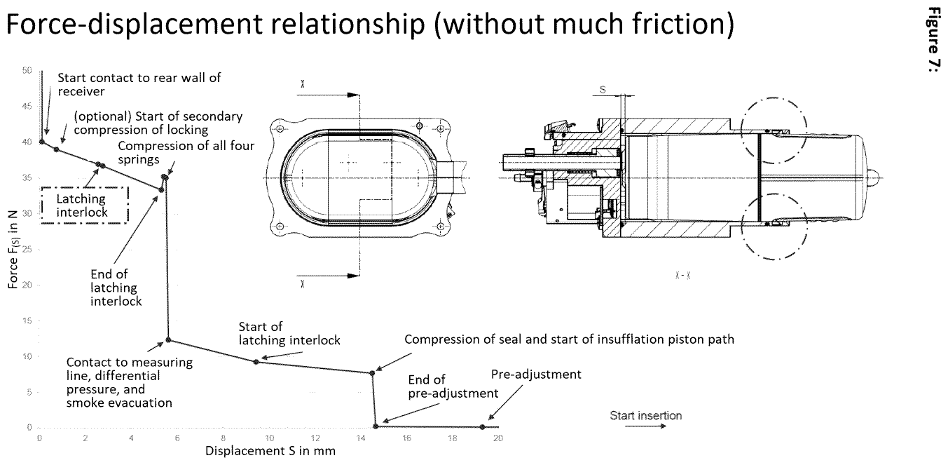

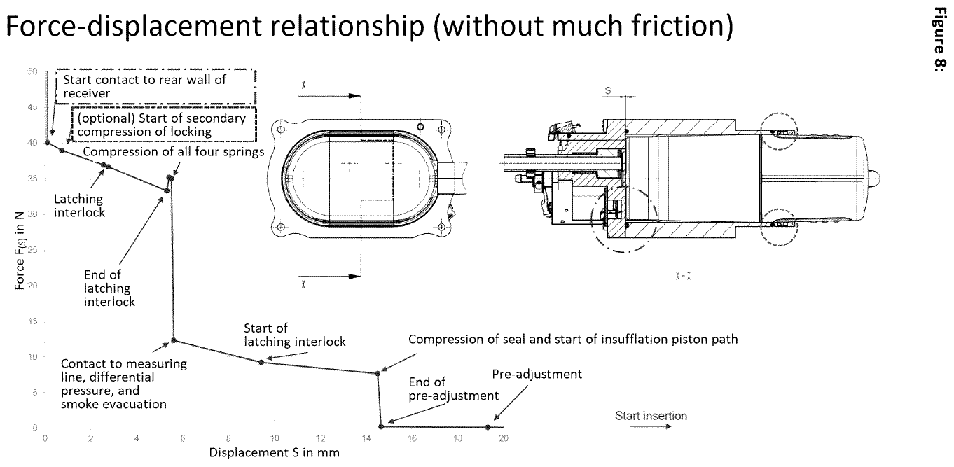

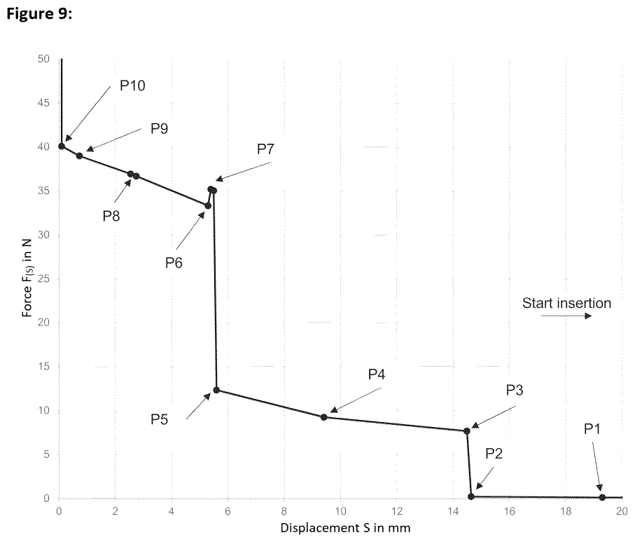

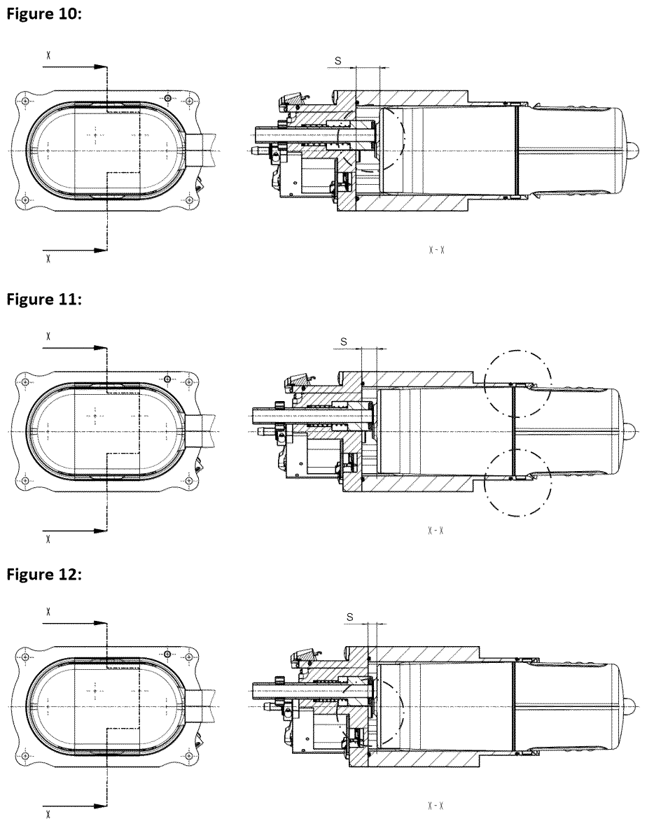

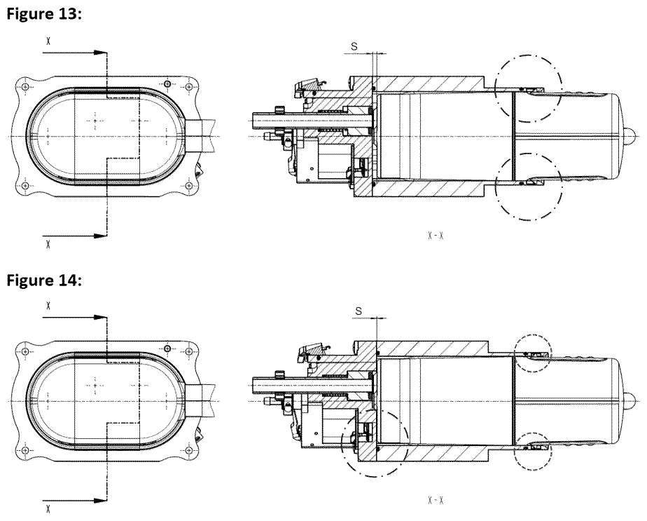

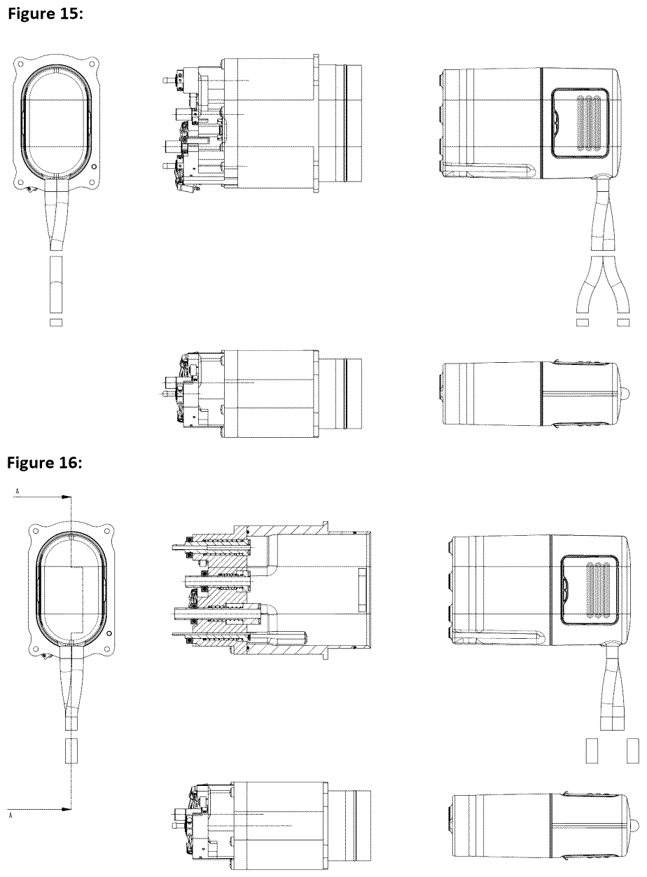

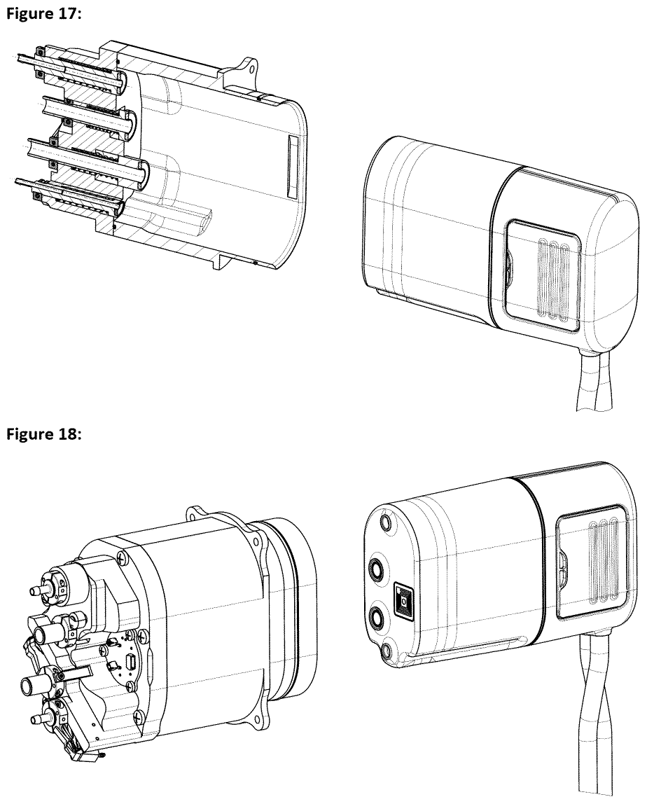

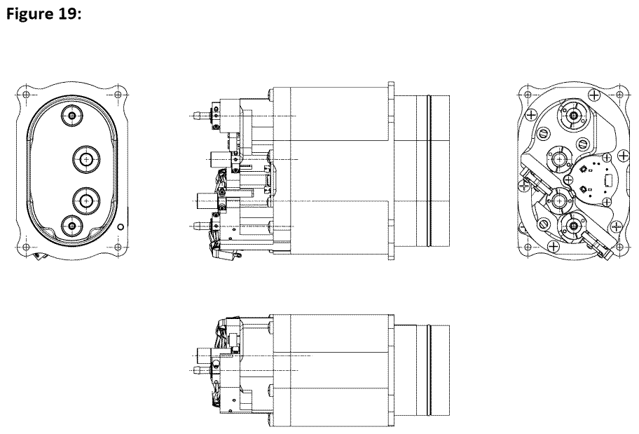

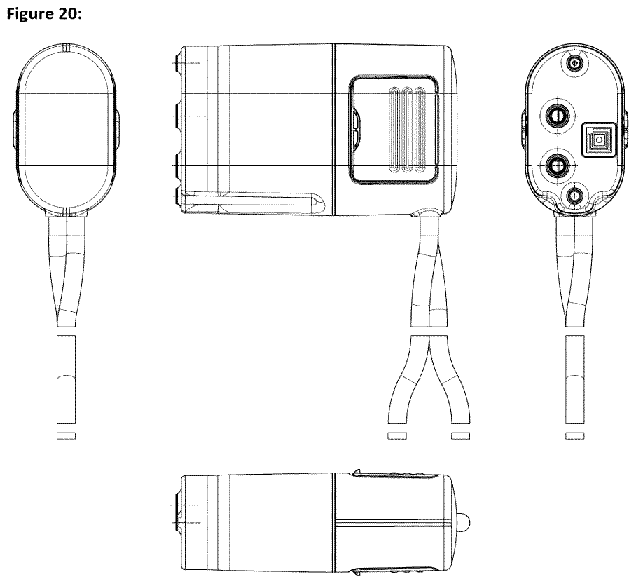

Top left: front view.

Top center, side view.

Top right: rear view.

Bottom: top view.

1. A tube connection device for connecting n tubes, through which a gas flows, with an insufflator, where n is one to five,
wherein a sleeve-shaped element is formed on the insufflator side,
wherein the sleeve-shaped element has n insufflation pistons as connecting elements in the region of the sleeve base,
wherein the n insufflation pistons as connecting elements protrude from the sleeve base by means of spring tension and can be pressed towards the sleeve base by the pressure from the tubes, whereby the surface of the insufflation pistons is flush with the sleeve base when the tube connection is made,
wherein a resilient sealing element is arranged between each connecting element and each tube, wherein one or more latching elements are arranged in the region of the sleeve opening, which ensure that the tubes engage and lock the tube connection,
wherein connecting elements are arranged on the tube side which are compatible with the connecting elements of the sleeve base,
wherein an element compatible with the sleeve-shaped element has n hollow cylinders as a tube set to which the n tubes are connected so as to ensure a fluid connection of all n tubes to the insufflator.

2. The tube connection device according to claim 1, wherein the number of tubes n is two to five.

3. The tube connection device of n tubes through which a gas flows with an insufflator according to claim 1, wherein the resilient sealing element is either a component of the insufflation flask as a connecting element or a component of the tube-side connecting elements.
